(19) United States
(12) Patent Application Publication
(10) Pub. No.: US 2026/0160750 A1
(43) Pub. Date: Jun. 11, 2026

(54) SYSTEMS AND METHODS FOR CETANE NUMBER RATING WITH ELECTRONIC FUEL INJECTION

(71) Applicant: CFR Engines Inc., Pewaukee, WI (US)

(72) Inventors: Kevin Nielson, Menomonee Falls, WI (US); Robert Sinur, Brookfield, WI (US)

(73) Assignee: CFR Engines Inc., Pewaukee, WI (US)

(21) Appl. No.: 19/183,228

(22) Filed: Apr. 18, 2025

Related U.S. Application Data
(60) Provisional application No. 63/636,431, filed on Apr. 19, 2024.

(51) Int. Cl. G01N 33/28; F02D 41/20; F02D 41/38; F02M 37/00

(52) U.S. Cl. CPC G01N 33/2829; F02D 41/20; F02D 41/38; F02M 37/0011

(57) ABSTRACT

An electronic fuel injection system for a cetane number rating system includes a fuel reservoir, a supply line, a drain line, an electrically-controlled fuel valve configured to selectively provide or inhibit fluid communication between the fuel reservoir and the supply line, an electrically-controlled drain valve configured to selectively provide or inhibit fluid communication between the fuel reservoir and the drain line, a low-pressure pump in fluid communication with the supply line, a high-pressure pump in fluid communication with an outlet of the low-pressure pump, and an electronic fuel injector in fluid communication with an outlet of the high-pressure pump. The high-pressure pump is configured to supply pressurized fuel from the fuel reservoir to the electronic fuel injector at a constant flow rate.

### CROSS-REFERENCE TO RELATED PATENT APPLICATIONS

This application claims the benefit of and priority to U.S. Provisional Application No. 63/636,431, filed on Apr. 19, 2024, the entire disclosure of which is hereby incorporated by reference herein.

### BACKGROUND

The cetane rating for a diesel fuel is typically represented by a cetane number scale that ranges from 0 to 100 and provides an indication of the diesel fuel's propensity to auto-ignite (e.g., the diesel fuel's ignition delay).

### SUMMARY

In some aspects, the present disclosure relates to an electronic fuel injection system for a cetane number rating system, the electronic fuel injection system including: a fuel reservoir; a supply line; a drain line; an electrically-controlled fuel valve configured to selectively provide or inhibit fluid communication between the fuel reservoir and the supply line; an electrically-controlled drain valve configured to selectively provide or inhibit fluid communication between the fuel reservoir and the drain line; a low-pressure pump in fluid communication with the supply line; a high-pressure pump in fluid communication with an outlet of the low-pressure pump; and an electronic fuel injector in fluid communication with an outlet of the high-pressure pump, wherein the high-pressure pump is configured to supply pressurized fuel from the fuel reservoir to the electronic fuel injector at a constant flow rate.

In some aspects, the present disclosure relates to a cetane number rating system, including: a cetane test engine including a piston arranged within a cylinder; an in-cylinder sensor configured to measure a pressure within a combustion chamber defined between the piston and the cylinder; an electronic fuel injection system including an injector driver and an electronic fuel injector arranged to inject fuel into the combustion chamber; and a human-machine interface in communication with the cetane test engine and the electronic fuel injection system, wherein the human-machine interface includes a user interface and a controller, the controller being configured to: determine a start of injection for the electronic fuel injector by adding a constant to a start of energization of a current waveform supplied by the injector driver to the electronic fuel injector; calculate a start of combustion based on a constant offset from a location of a maximum pressure rise rate of the pressure within the combustion chamber measured by the in-cylinder sensor; and determine an ignition delay based on a difference between the start of combustion and the start of injection.

In some aspects, the present disclosure relates to a method for determining a cetane number of a sample diesel fuel, the method including: opening, in response an input to a user interface, a fuel valve to supply a sample diesel fuel to a supply line; pumping the sample diesel fuel from the supply line to an electronic fuel injector at a constant flow rate; instructing, via an injector driver, the electronic fuel injector to inject the sample diesel fuel into a cetane test engine; measuring, via a current sensor, a current waveform supplied by the injector driver to the electronic fuel injector; calculating a start of injection by: determining a start of energization from the current waveform; and adding a constant to the start of energization; measuring, via an in-cylinder sensor, a pressure within a combustion chamber of the cetane test engine; calculating a start of combustion in the cetane test engine based on a constant offset from a location of a maximum pressure rise rate of the pressure measured by the in-cylinder sensor; calculating an ignition delay based on a difference between the start of combustion and the start of injection; turning a handwheel on the cetane test engine until the ignition delay becomes 13 crank angle degrees plus or minus 0.1 crank angle degrees; and recording a position of the handwheel when the ignition delay is 13 crank angle degrees plus or minus 0.1 crank angle degrees.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a perspective view of a cetane number rating system, according to an exemplary embodiment;

FIG. 2 is a schematic illustration of a combustion chamber within a cetane test engine of the cetane number rating system of FIG. 1;

FIG. 3 is a block diagram of the cetane test engine of the cetane number rating system of FIG. 1;

FIG. 4 is a schematic illustration of an electronic fuel injection system of the cetane number rating system of FIG. 1;

FIG. 5 is a schematic illustration of an injector nozzle of the electronic fuel injection system of FIG. 4;

FIG. 6 is a schematic illustration of a control system of the cetane number rating system of FIG. 1;

FIG. 7A is an image of a main screen on a user interface of the cetane number rating system of FIG. 1;

FIG. 7B is an image of a cetane test screen on a user interface of the cetane number rating system of FIG. 1;

FIG. 8 is an image of a fuel screen on the user interface of the cetane number rating system of FIG. 1;

FIG. 9 is an image of a graph screen on the user interface of the cetane number rating system of FIG. 1;

FIG. 10 is a graph illustrating start of injection, ignition delay, and start of combustion calculations using the cetane number rating system of FIG. 1; and

FIG. 11 is a flowchart outlining the steps in a cetane number procedure or method using the cetane number rating system of FIG. 1.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The standard test method or procedure for determining a cetane number of diesel fuel is governed by ASTM D613. The conventional test setup for conducting ASTM D613 includes a mechanical fuel system that delivers diesel fuel to a single-cylinder test engine. The mechanical fuel system includes a plunger/barrel injection pump that is gravity fed and subject to changes in the delivery rate as a function of the fuel level in the fuel bowl feeding the injection pump. In addition, the injection timing (e.g., start of injection (SOI)), the injection duration, and the fuel flow rate are manually adjusted, which introduces the potential for the test results to be operator dependent and reduces precision. Further, the injection timing, injection duration, and injection quantity all vary due to changing fluid dynamics within the system, and differences in the physical properties of the test fuels and the reference fuels amplify these inconsistent fluid dynamics. For example, the injection pump builds from zero pressure to injection pressure for each injection and increases the fluid dynamic irregularities within the mechanical injection system and reduces precision.

The accuracy of the cetane number determined by ASTM D613 and the precision (e.g., repeatability) with which the cetane number procedure is carried out have a significant impact on automotive/research industries and the fuel supply chain (e.g., refineries). For example, producing accurate and precise cetane number results enables automotive/research industries to effectively test new diesel fuel blends. Also, the fuel supply chain business is heavily dependent on the accuracy of the cetane number so refined fuels may be accurately categorized and sold according to the fuel quality (i.e., cetane number) determined by ASTM D613.

The systems and methods of the present disclosure provide an electronic fuel injection system that greatly improves the precision, accuracy, and operator efficiency of the ASTM D613 cetane number procedure. Specifically, the electronic fuel injection system of the present disclosure is not as sensitive to the variations in fluid dynamics driven by the difference in physical properties between the fuels, which improves the precision, accuracy, and operator efficiency of the ASTM D613 cetane number procedure. In some embodiments, the electronic fuel injection system includes an electronically-driven high-pressure pump that supplies high-pressure fuel to an electronic fuel injector. The electronic control of the high-pressure pump and the fuel injector enables the fuel flow rate, the SOI, the injection duration, and the injection quantity to be set electronically and held approximately constant during the cetane test procedure, which removes the need for manual adjustment of these parameters required in the conventional mechanical fuel system and improves precision. Additionally, the electronic fuel injector provides superior fuel atomization and air-fuel mixing during the combustion cycle, which provides cleaner combustion, more consistent equilibrium conditions, lower cycle-to-cycle variations, and longer maintenance intervals.

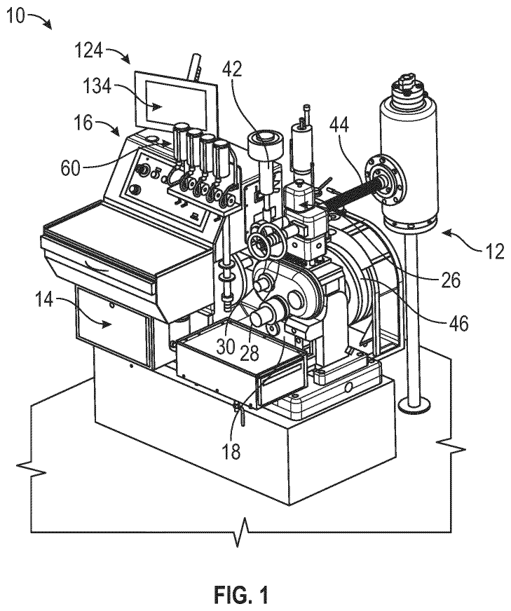

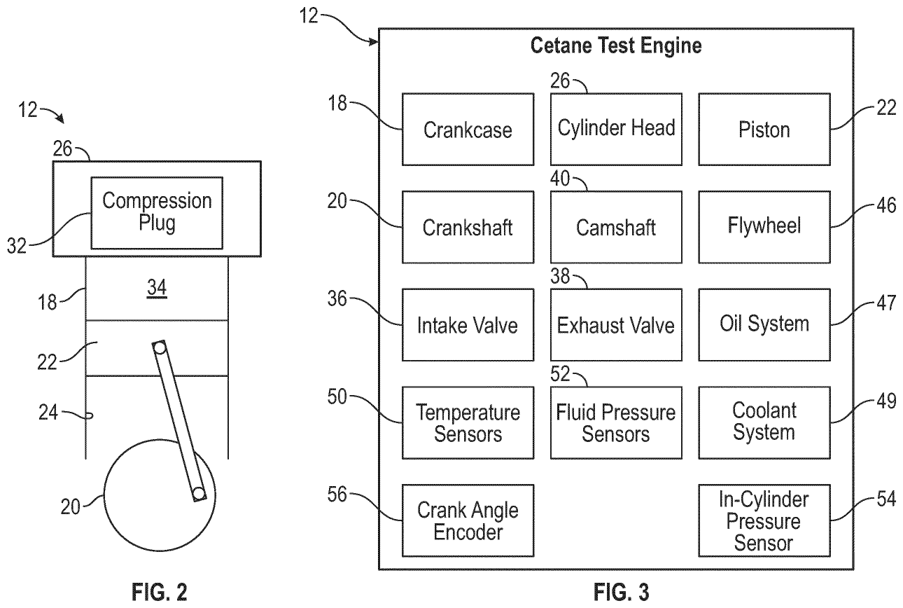

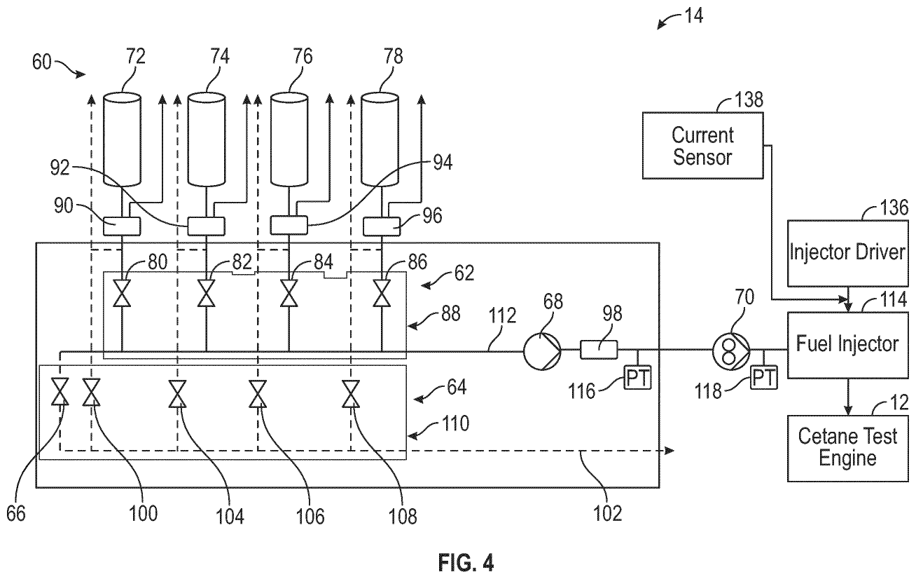

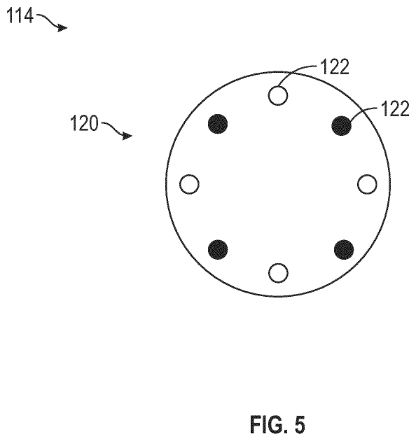

FIGS. 1-6 show a cetane number rating system 10 that is used to determine a cetane number of a sample diesel fuel relative to two reference diesel fuels according to ASTM D613. The cetane number rating system 10 includes a cetane test engine 12, an electronic fuel injection (EFI) system 14, and a human-machine interface (HMI) 16. In some embodiments, the cetane test engine 12 is a single-cylinder, four-stroke cycle, variable compression ratio, indirect injected diesel engine. In some embodiments, the cetane test engine 12 is a model F5 engine unit manufactured by CFR Engines Inc.

With specific reference to FIGS. 1-3, the cetane test engine 12 includes a crankcase 18 that encloses a crankshaft 20. The crankshaft 20 is rotatably coupled to a piston 22 so that rotation of the crankshaft 20 results in reciprocal motion of the piston 22 within a cylinder bore 24 defined within the crankcase 18. A cylinder head 26 is coupled to a top side of the crankcase 18. The cylinder head 26 enables the cetane test engine 12 to define a variable compression ratio by including a handwheel 28 and a locking wheel 30. The handwheel 28 extends from the cylinder head 26 and is rotatably coupled to a variable compression plug 32. The locking wheel 30 is configured to selectively lock the position of the variable compression plug 32, and rotation of the handwheel 28 (when the locking wheel 30 unlocks the variable compression plug 32) adjust the compression ratio of the cetane test engine 12. A micrometer is coupled to the handwheel 28 and includes a scale that extends from 0.500 to 3.000 and is inversely related to the compression ratio (low handwheel readings correspond to high compression ratio conditions, while high handwheel readings correspond to low compression ratio conditions).

In general, the volume within the cylinder bore 24 between the piston 22 and the cylinder head 26 is defined as a combustion chamber 34 (e.g., a main combustion chamber). In some embodiments, the cylinder head 26 includes a precombustion chamber within which the variable compression plug 32 is arranged. In some embodiments, an intake valve 36 and an exhaust valve 38 are housed within the cylinder head 26. The intake valve 36 is configured to selectively open and provide intake air from an intake conduit 42 to the combustion chamber 34 based on the timing governed by rotation of a camshaft 40. The exhaust valve 38 is configured to selectively open and provide exhaust gases from the combustion chamber 34 to an exhaust conduit 44 based on the timing governed by rotation of the camshaft 40. The camshaft 40 is rotatably coupled to the crankshaft 20 (e.g., via a geartrain) so that two rotations of the crankshaft 20 result in one rotation of the camshaft 40 and the cetane test engine 12 operates on a four-stroke engine cycle (intake stroke, compression stroke, power stroke, exhaust stroke).

In some embodiments, the cetane test engine 12 includes a flywheel 46 rotatably coupled to the crankshaft 20 so that rotation of the crankshaft 20 results in rotation of the flywheel 46. In some embodiments, the cetane test engine 12 includes an oil system 47 (e.g., an oil pump and an oil filter) that provides lubricating oil to various components within the crankcase 18 and the cylinder head 26. In some embodiments, the cetane test engine 12 includes a coolant system 49 (e.g., a static water jacket, a coolant pump, etc., that supplies coolant to one or more coolant passages within the crankcase 18 and/or the cylinder head 26, and a heat exchanger).

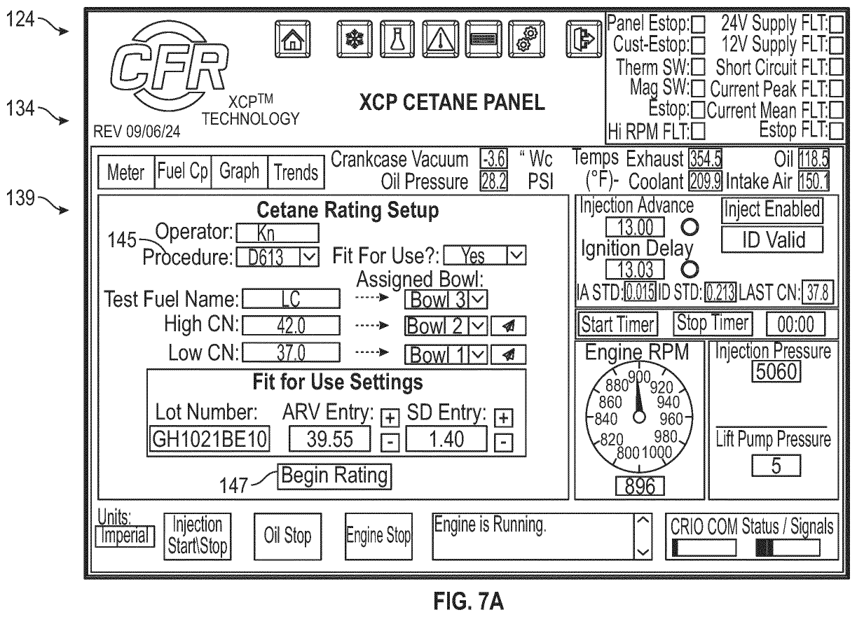

In general, the cetane test engine 12 includes a plurality of instrumentation sensors that are configured to measure engine operating parameters and communicate the engine operating parameters to the HMI 16 (see, e.g., FIG. 6). For example, the cetane test engine 12 includes a plurality of temperature sensors 50, a plurality of fluid pressure sensors 52, an in-cylinder sensor 54, and a crank angle encoder 56. In some embodiments, the temperature sensors 50 include an oil temperature sensor, an intake air temperature sensor, an exhaust gas temperature sensor, and a coolant temperature sensor. In some embodiments, the fluid pressure sensors 52 include an oil pressure sensor, a crankcase pressure sensor, a coolant pressure sensor, and an intake air pressure sensor. The in-cylinder sensor 54 may be mounted to the cylinder head 26 and extend into the combustion chamber 34 (or the precombustion chamber) so that the in-cylinder sensor 54 measures a pressure within the combustion chamber 34, or a quantity that is correlated to the pressure within the combustion chamber 34, while the cetane test engine 12 is running (e.g., during the four-stoke cycle). In some embodiments, the in-cylinder sensor 54 may directly measure the pressure within the combustion chamber 34 (e.g., the in-cylinder sensor 54 may be a pressure transducer). In some embodiments, the in-cylinder sensor 54 may measure a quantity (e.g., voltage) that is correlated to the pressure within the combustion chamber 34. The crank angle encoder 56 is configured to measure a rotational position of the crankshaft 20 (e.g., crank angle degrees), which corresponds to a position of the piston 22 along the cylinder bore 24 during the four-stroke cycle. In some embodiments, the crank angle encoder 56 is an optical encoder configured to measure at 4×2500 (counts/rev). In some embodiments, the crank angle encoder 56 is configured to measure engine speed (in revolutions per minute (RPM)) in addition to measuring the crank angle position.

In general, the EFI system 14 is configured to selectively inject the sample diesel fuel or one of the reference diesel fuels to the cetane test engine 12 at a predetermined time (SOI) and for a predetermined duration (e.g., a predetermined fuel quantity). With specific reference to FIGS. 1, 4, and 5, the EFI system 14 includes a plurality of fuel reservoirs, cylinders, or tanks 60, a plurality of fuel valves 62, a plurality of drain valves 64, a flush valve 66, a low-pressure pump 68, and a high-pressure pump 70. In the illustrated embodiment, the EFI system 14 includes four fuel reservoirs 60 and a corresponding number of fuel valves 62 and drain valves 64. For example, the EFI system 14 includes a first fuel reservoir 72, a second fuel reservoir 74, a third fuel reservoir 76, and a fourth fuel reservoir 78. A first fuel valve 80 is in fluid communication with and arranged downstream of the first fuel reservoir 72 and is configured to selectively provide or inhibit fluid communication between the first fuel reservoir 72 and the low-pressure pump 68. A second fuel valve 82 is in fluid communication with and arranged downstream of the second fuel reservoir 74 and is configured to selectively provide or inhibit fluid communication between the second fuel reservoir 74 and the low-pressure pump 68. A third fuel valve 84 is in fluid communication with and arranged downstream of the third fuel reservoir 76 and is configured to selectively provide or inhibit fluid communication between the third fuel reservoir 76 and the low-pressure pump 68. A fourth fuel valve 86 is in fluid communication with and arranged downstream of the fourth fuel reservoir 78 and is configured to selectively provide or inhibit fluid communication between the fourth fuel reservoir 78 and the low-pressure pump 68. In some embodiments, the EFI system 14 may include more or less than four fuel reservoirs 60 and a corresponding number of fuel valves 62 and drain valves 64. In some embodiments, each of the fuel valves 62 (e.g., the first fuel valve 80, the second fuel valve 82, the third fuel valve 84, and the fourth fuel valve 86) is a solenoid-operated valve (e.g., a solenoid-operated ball valve, a solenoid-operated spool valve, a solenoid-operated on/off valve, etc.). In some embodiments, each of the fuel valves 62 is arranged within a fuel supply manifold 88.

A fuel filter is arranged between each of the fuel reservoirs 60 and the corresponding fuel valve 62 connected to the fuel reservoir 60. For example, a first fuel filter 90 is arranged between the first fuel reservoir 72 and the first fuel valve 80, a second fuel filter 92 is arranged between the second fuel reservoir 74 and the second fuel valve 82, a third fuel filter 94 is arranged between the third fuel reservoir 76 and the third fuel valve 84, and a fourth fuel filter 96 is arranged between the fourth fuel reservoir 78 and the fourth fuel valve 86. In some embodiments, each of the first fuel filter 90, the second fuel filter 92, the third fuel filter 94, and the fourth fuel filter 96 is a preliminary fuel filter that defines a first porosity or is designed to filter particles having a first particle size (e.g., about 10 microns). A secondary fuel filter 98 is arranged downstream of the low-pressure pump 68. In some embodiments, the secondary fuel filter 98 defines a second porosity or is designed to filter particles having a second particle size that is smaller than the first particle size (e.g., about 2 microns).

Each of the drain valves 64 is connected between one of the fuel reservoirs 60 and a corresponding one of the fuel filters. For example, a first drain valve 100 is connected between the first fuel filter 90 and the first fuel valve 80 and is configured to selectively provide or inhibit fluid communication between a drain line or conduit 102 and both of the first fuel reservoir 72 and the first fuel filter 90. A second drain valve 104 is connected between the second fuel filter 92 and the second fuel valve 82 and is configured to selectively provide or inhibit fluid communication between the drain line 102 and both of the second fuel reservoir 74 and the second fuel filter 92. A third drain valve 106 is connected between the third fuel filter 94 and the third fuel valve 84 and is configured to selectively provide or inhibit fluid communication between the drain line 102 and both of the third fuel reservoir 76 and the third fuel filter 94. A fourth drain valve 108 is connected between the fourth fuel filter 96 and the fourth fuel valve 86 and is configured to selectively provide or inhibit fluid communication between the drain line 102 and both of the fourth fuel reservoir 78 and the fourth fuel filter 96. In some embodiments, each of the drain valves 64 (e.g., the first drain valve 100, the second drain valve 104, the third drain valve 106, and the fourth drain valve 108) is a solenoid-operated valve (e.g., a solenoid-operated ball valve, a solenoid-operated spool valve, a solenoid-operated on/off valve, etc.). In some embodiments, each of the drain valves 64 is arranged within a drain manifold 110.

The low-pressure pump 68 is configured to receive fuel from one of the fuel reservoirs 60 via opening of the corresponding fuel valve 62. For example, an inlet of the low-pressure pump 68 is in fluid communication with a fuel supply line or conduit 112 and an outlet of each of the first fuel valve 80, the second fuel valve 82, the third fuel valve 84, and the fourth fuel valve 86 is in fluid communication with the fuel supply line 112. In this way, for example, opening one of the first fuel valve 80, the second fuel valve 82, the third fuel valve 84, and the fourth fuel valve 86 supplies the fuel within the corresponding one of the first fuel reservoir 72, the second fuel reservoir 74, the third fuel reservoir 76, or the fourth fuel reservoir 78 to the fuel supply line 112 and to the low-pressure pump 68. In some embodiments, the low-pressure pump 68 is electrically powered and configured to output fuel at a flow rate of about 11 milliliters per min (mL/min) at a pressure of 0.5 bar and at 24 VDC.

The flush valve 66 is connected to the fuel supply line 112 downstream of each of the fuel valves 62 and is configured to selectively provide or inhibit fluid communication between the fuel supply line 112 and the drain line 102. In this way, for example, selectively opening of the flush valve 66 provides fluid communication between the fuel supply line 112 and the drain line 102, which drains the fuel in the fuel supply line 112. In some embodiments, the flush valve 66 is a solenoid-operated valve (e.g., a solenoid-operated ball valve, a solenoid-operated spool valve, a solenoid-operated on/off valve, etc.).

The high-pressure pump 70 is arranged downstream of the low-pressure pump 68. Specifically, an outlet of the low-pressure pump 68 is in fluid communication with an inlet of the high-pressure pump 70. In some embodiments, the high-pressure pump 70 is an electrically-controlled positive displacement fuel pump that is configured to operate at a max pressure of 500 bar. In some embodiments, the high-pressure pump 70 is configured to output high-pressure fuel to a fuel injector 114 at a constant fuel flow rate of about 11 mL/min plus or minus 0.1 mL/min, or about 11 mL/min plus or minus 0.2 mL/min, or about 11 mL/min plus or minus 0.3 mL/min, or about 11 mL/min plus or minus 0.4 mL/min, or about 11 mL/min plus or minus 0.5 mL/min. The fuel flow rate of about 11 mL/min provides the surprising and unexpected effect of approximately matching the combustion characteristics (e.g., pressure rise rate, peak pressure, exhaust temperature, etc.) of the mechanical fuel system used in ASTM D613, where the fuel flow rate is manually adjusted to 13 mL/min. Accordingly, the incorporation of the EFI system 14 into the cetane rating number rating system 10 surprisingly provides similar combustion characteristics using a lower fuel flow rate, and the fuel flow rate is electronically commanded and held constant by the high-pressure pump 70, which eliminates the requirement for an operator to manually adjust and maintain the fuel flow rate and improves precision during a cetane number procedure (ASTM D613).

The EFI system 14 includes a first pressure sensor 116 arranged between the low-pressure pump 68 and the high-pressure pump 70, and a second pressure sensor 118 arranged between the high-pressure pump 70 and the fuel injector 114 (e.g., at an outlet of the high-pressure fuel pump 70). Specifically, the first pressure sensor 116 is arranged between the secondary fuel filter 98 and the high-pressure pump 70. In general, the first pressure sensor 116 is configured to monitor the output pressure from the low-pressure pump 68 and monitor a cleanliness of the secondary fuel filter 98. For example, if the pressure measured by the first pressure sensor 116 falls below (e.g., is less than or equal to) a predetermined threshold, the secondary fuel filter 98 may require maintenance and an indication may be provided to the operator via the HMI 16. The second pressure sensor 118 is configured to monitor the output pressure from the high-pressure pump 70 that is supplied to the fuel injector 114 (e.g., the injection pressure) and monitor the health of the high-pressure pump 70. For example, if the pressure measured by the second pressure sensor 118 falls below (e.g., is less than or equal to) a predetermined lower threshold, the high-pressure pump 70 may require maintenance and an indication may be provided to the operator via the HMI 16. If the pressure measured by the second pressure sensor 118 raises above (e.g., is greater than or equal to) a predetermined upper threshold, the fuel injector 114 may be dirty or clogged and require maintenance, and an indication may be provided to the operator via the HMI 16.

In some embodiments, the fuel injector 114 is an electronic solenoid-style fuel injector with a modified nozzle. In some embodiments, the fuel injector 114 is driven by an injector driver 136 that is configured to output a current waveform to the fuel injector 114 that control the start of injection and injection duration of the fuel injector 114. A current sensor 138 is configured to measure the current waveform supplied by the injector driver 136 to the fuel injector 114 as a function of crank angle degrees.

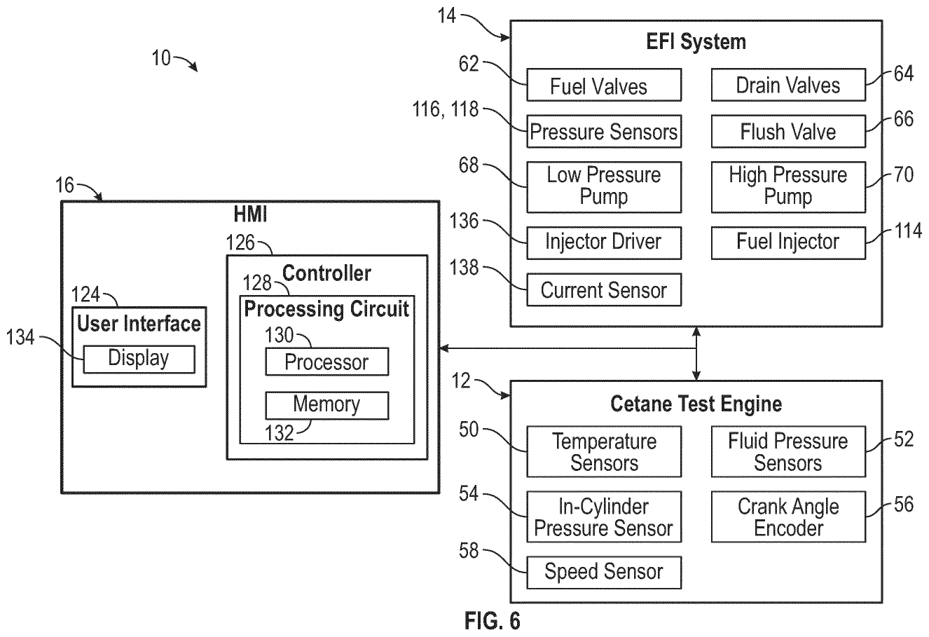

With specific reference to FIG. 5, the fuel injector 114 includes a fuel tip 120 having a plurality of nozzle holes 122 extending through the fuel tip 120 through which the high-pressure fuel is selectively injected into the cetane test engine 12 (e.g., into the combustion chamber 34 or the precombustion chamber) via operation of the solenoid within the fuel injector 114. In some embodiments, a portion of the nozzle holes 122 are plugged (e.g., welded closed) so that fuel cannot be sprayed from the plugged nozzle holes 122 (indicated as filled in circles in FIG. 5). Specifically, in the illustrated embodiment, the fuel tip 120 includes eight nozzle holes 122 and every other nozzle hole 122 is plugged (e.g., every pair of circumferentially-adjacent nozzles holes 122 includes one open nozzle hole 122 and one plugged nozzle hole 122). As such, the fuel injector 114 is modified to reduce the number of nozzle holes 122 from eight down to four. Plugging a portion of the nozzle holes 122 provides the surprising and unexpected effect of allowing the EFI system 14 to lengthen the injection duration, which approximately matches the combustion characteristics (e.g., pressure rise rate, peak pressure, exhaust temperature, etc.) of the mechanical fuel system used in ASTM D613. In addition, the fuel injector 114 provides superior fuel atomization and air-fuel mixing during the combustion cycle (when compared to the mechanical fuel system used in ASTM D613), which provides cleaner combustion, more consistent equilibrium conditions, lower cycle-to-cycle variations, reduced maintenance intervals, and more precise cetane number ratings. In some embodiments, the fuel injector 114 includes a particular number of open nozzle holes 122 that satisfy the injection pressure and injection duration requirements of the cetane number procedure described herein (e.g., 2 holes, 3 holes, 4 holes, 5holes, etc.).

In general, the EFI system 14 is compatible to operate with various diesel fuels including ASTM D975, biodiesel blends, hydrotreated vegetable oils, gas-to-liquid fuel, primary cetane reference fuels (e.g., n-hexadecane, heptamethylnonane (HMN), pentamethylheptane (PMH), etc.), secondary cetane reference fuels, diesel fuels with a density between about 700 kilograms per meter cubed (kg/m³)@15° C. and about 900 kg/m³@15° C., and/or fuels with a viscosity between about 1.0 centistokes (cSt)@40° C. and about 5.0 cSt@40° C.

Turning to FIGS. 1 and 6-9, the incorporation of the EFI system 14 enables electronic control and calculation of the parameters used during a cetane number procedure (ASTM D613) via the HMI 16. The HMI 16 includes a user interface 124 and a controller 126 in communication with the user interface 124, the cetane test engine 12, and the EFI system 14. The controller 126 includes a processing circuit 128 having a processor 130 and memory 132. The processing circuit 128 can be communicably connected to a communications interface such that the processing circuit 128 and the various components thereof can send and receive data via the communications interface. The processor 130 can be implemented as a general-purpose processor, an application specific integrated circuit (“ASIC”), one or more field programmable gate arrays (“FPGAs”), a group of processing components, or other suitable electronic processing components.

The memory 132 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. The memory 132 can be or include volatile memory or non-volatile memory. The memory 132 can include database components, object code components, script components, or any other type of information structure for supporting the various activities and information structures described in the present application. According to some embodiments, the memory 132 is communicably connected to the processor 130 via the processing circuit 128 and includes computer code for executing (e.g., by the processing circuit 128 and/or the processor 130) one or more processes, methods, or procedures described herein.

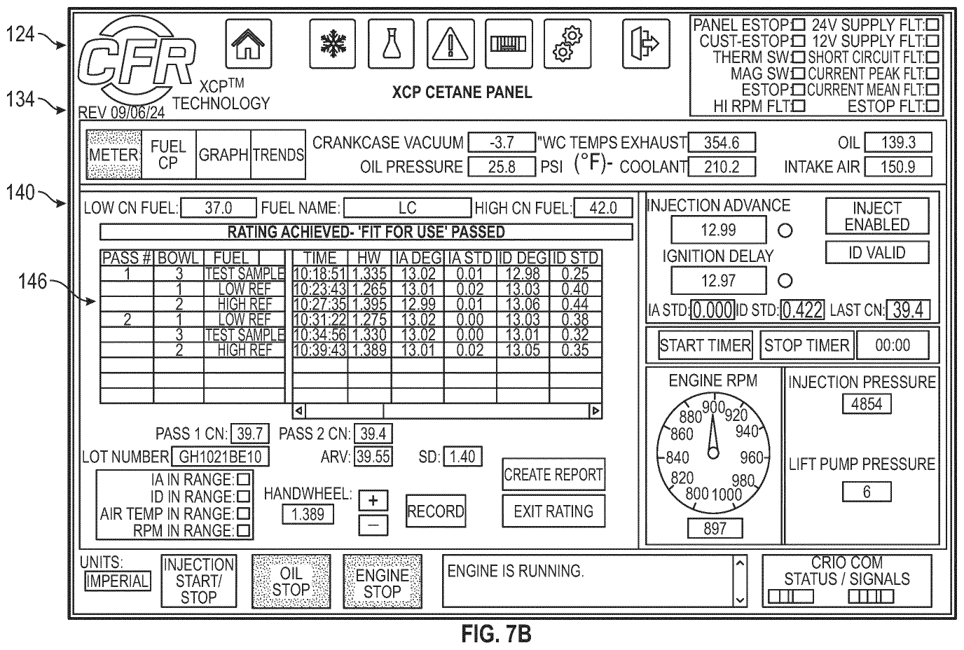

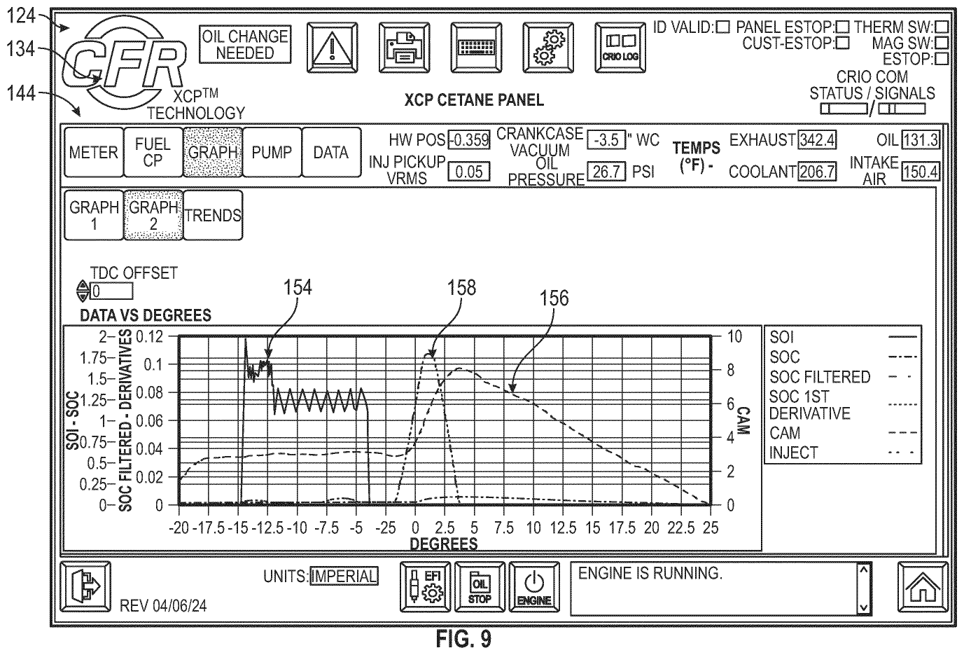

In general, the controller 126 is in communication with the cetane test engine 12, the EFI system 14, and the user interface 124 and configured to receive various data inputs from the cetane test engine 12, the EFI system 14, and the user interface 124. For example, the controller 126 is configured to receive data inputs from the temperature sensors 50, the fluid pressure sensors 52, the in-cylinder sensor 54, and the crank angle encoder 56 of the cetane test engine 12. In some embodiments, the controller 126 receives data inputs from an exhaust gas temperature sensor, a coolant temperature sensor, an oil temperature sensor, and an intake air temperature sensor of the temperature sensors 50 and instructs the user interface 124 to display the corresponding temperatures on a display 134 of the user interface 124 (see, e.g., FIGS. 7A-9). In some embodiments, the controller 126 receives data inputs from an oil pressure sensor and a crankcase pressure sensor of the fluid pressure sensors 52 and instructs the user interface 124 to display the corresponding pressures on the display 134 (see, e.g., FIGS. 7A-9). In some embodiments, the controller 126 receives a data inputs from the in-cylinder sensor 54 and the crank angle encoder 56 and instructs the user interface 124 to display the pressure within the combustion chamber 34 as a function of crank angle degrees on the display 134 (see, e.g., FIG. 9). As described herein, the pressure measured by the in-cylinder sensor 54 as a function of crank angle degrees is utilized by the controller 126 to calculate a start of combustion (SOC) that is used in an ignition delay calculation. In some embodiments, the controller 126 receives a data input from the crank angle encoder 56 and instructs the user interface 124 to display the speed of the cetane test engine 12 on the display 134 (see, e.g., FIG. 7A-9).

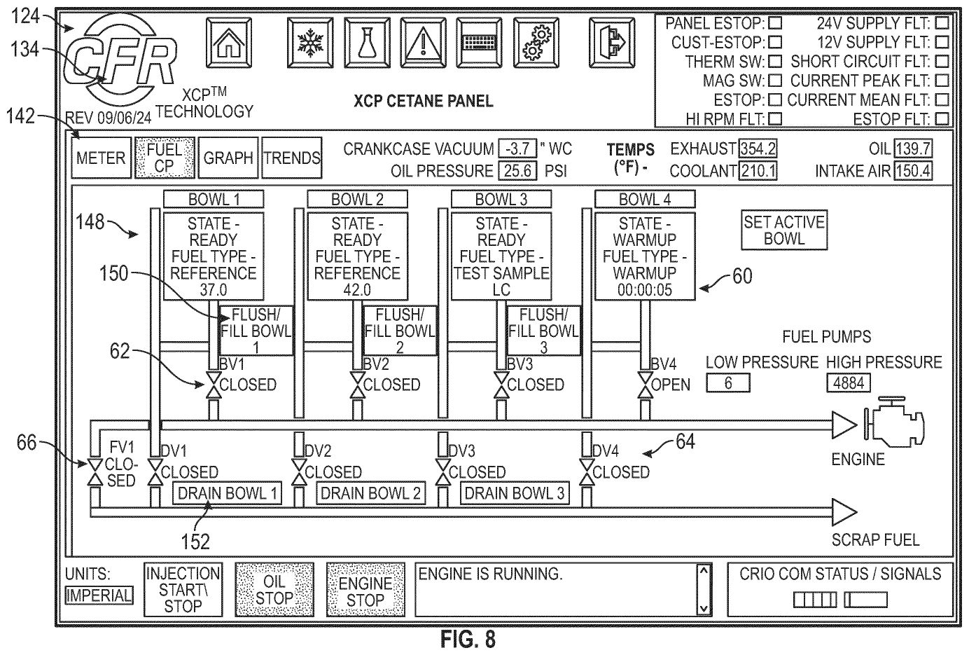

The controller 126 is in communication with and configured to receive data inputs from the first pressure sensor 116 and the second pressure sensor 118. The controller 126 is configured to instruct the user interface 124 to display the corresponding pressures on the display 134 (see, e.g., FIG. 8). As described herein, the data from the first pressure sensor 116 and the second pressure sensor 118 may be used to monitor a health or, determine if maintenance is required, for the low-pressure pump 68 and the high-pressure pump 70, respectively. In some embodiments, the controller 126 is configured to monitor the data from the first pressure sensor 116 and determine if the pressure measured by the first pressure sensor 116 falls below (e.g., is less than or equal to) the predetermined threshold. If the pressure falls below the predetermined threshold, the controller 126 may instruct the user interface 124 to provide an indication (e.g., a visual indication on the display 134, a text indication on the display 134 and/or an audible indication from a speaker of the user interface 124) that maintenance is required for the secondary fuel filter 98. Similarly, the controller 126 is configured to monitor the data from the second pressure sensor 118 and determine if the pressure measured by the second pressure sensor 118 falls below (e.g., is less than or equal to) the predetermined lower threshold. If the pressure falls below the predetermined lower threshold, the controller 126 may instruct the user interface 124 to provide an indication (e.g., a visual indication on the display 134, a text indication on the display 134 and/or an audible indication from a speaker of the user interface 124) that maintenance is required for the high-pressure pump 70. Additionally, the controller 126 is configured to monitor the data from the second pressure sensor 118 and determine if the pressure measured by the second pressure sensor 118 raises above (e.g., is greater than or equal to) the predetermined upper threshold. If the pressure raises above the predetermined upper threshold, the controller 126 may instruct the user interface 124 to provide an indication (e.g., a visual indication on the display 134, a text indication on the display 134 and/or an audible indication from a speaker of the user interface 124) that fuel injector 114 needs to be cleaned.

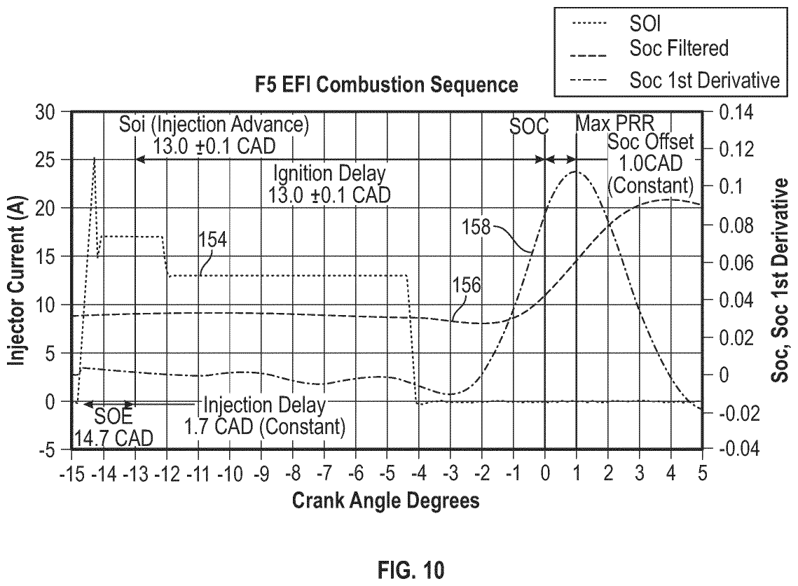

As described herein, the fuel injector 114 may be a solenoid-style fuel injector. The injector driver 136 is configured to send a current waveform to the solenoid of the fuel injector 114 that controls the opening and closing of the fuel injector 114. The current sensor 138 is configured to measure the current waveform supplied from the injector driver 136 to the fuel injector 114. In some embodiments, the controller 126 is in communication with and receives data input from the current sensor 138, and is configured to instruct the user interface 124 to display a graph of the current waveform supplied to the fuel injector 114 as a function of crank angle degree (see, e.g., FIG. 9) on the display 134. With the controller 126 receiving data inputs from the current sensor 138 and the crank angle encoder 56, the current waveform supplied to the fuel injector 114 is known as a function of crank angle degrees. As such, the controller 126 is configured to calculate the start of energization (SOE) based on the current waveform measured by the current sensor 138 as a function of crank angle degree. In general, the SOE is defined as the crank angle degree where the current waveform begins (e.g., increases above a threshold current value). The start of injection (SOI) is calculated, according to the cetane number procedure of the present disclosure, as SOE plus a constant value (e.g., SOI=SOE+Constant). In general, the constant value (e.g., injection delay in FIG. 10) is added to the SOE to account for fuel-to-fuel variations in the delay between when the current waveform starts (SOE) and when fuel pressure drops to indicate fuel is flowing from the fuel injector 114. Utilizing this constant value to account for the fuel-to-fuel variations avoids the complexities associated with using a delicate pressure sensor to detect when injection pressure drops after SOE and makes the SOI calculation computationally more efficient.

In addition to the various data inputs provided to the controller 126, the controller 126 is configured to output various control signals to control operation of the cetane test engine 12 and the EFI system 14. For example, the controller 126 is in communication with the fuel valves 62, the drain valves 64, the flush valve 66, the low-pressure pump 68, the high-pressure pump 70, and the injector driver 136. In some embodiments, the controller 126 is configured to selectively supply control signals to the fuel valves 62 (e.g., the first fuel valve 80, the second fuel valve 82, the third fuel valve 84, and the fourth fuel valve 86) to control the opening and closing thereof. For example, to supply fuel from the first fuel reservoir 72 to the fuel injector 114, the controller 126 may instruct the first fuel valve 80 to actuate from a closed position to an open position, which provides fluid communication between the first fuel reservoir 72 and fuel injector 114 via the fuel supply line 112, the low-pressure pump 68, and the high-pressure pump 70.

In some embodiments, the controller 126 is configured to selectively supply control signals to the drain valves 64 (e.g., the first drain valve 100, the second drain valve 104, the third drain valve 106, and the fourth drain valve 108) to control the opening and closing thereof. For example, to drain fuel from the first fuel reservoir 72 and the first fuel filter 90, the controller 126 may instruct the first fuel valve 80 to close and instruct the first drain valve 100 to open, which connects the first fuel reservoir 72 and the first fuel filter 90 to the drain line 102. Similarly, the controller 126 is configured to selectively supply a control signal to the flush valve 66 to control the opening and closing thereon. In some embodiments, the controller 126 is configured to automatically instruct the flush valve 66 to actuate or move from a closed position to an open position when the fuel is changed from one of the fuel reservoirs 60 to another of the fuel reservoirs 60. For example, in response to the fuel reservoir that is supplying fuel to the cetane test engine 12 being changed to a different fuel reservoir, the controller 126 may instruct the flush valve 66 to open for a predetermined amount of time (e.g., about 2 seconds, about 3 seconds, about 4 seconds, or about 5 seconds) to drain the fuel in the fuel supply line 112 and reduce the amount of time required to clear the EFI system 14 and the cetane test engine 12 of the previous fuel, which reduces the operator downtime in between tests.

In some embodiments, the controller 126 is configured to instruct the low-pressure pump 68 to supply fuel from the fuel supply line 112 to the high-pressure pump 70 at an approximately constant fuel flow rate (e.g., about 11 mL/min@1 bar). In some embodiments, the controller 126 is configured to instruct the high-pressure pump 70 to supply high-pressure fuel to the fuel injector 114 at an approximately constant flow rate (e.g., about 11 mL/min). In some embodiments, the controller 126 is configured to selectively supply a control signal to the injector driver 136 that, in turn, instructs the injector driver 136 to supply the current waveform to the fuel injector 114 at a particular crank angle degree for a particular injection duration. In general, the controller 126 is configured to instruct the injector driver 136 to send the current waveform to the fuel injector at a constant crank angle degree for a constant injection duration, which eliminates the need for an operator to adjust these parameters for different fuels (with the mechanical fuel system described herein) and improves the precision of the cetane number procedure. In some embodiments, the injection duration is about 2 milliseconds (ms) plus or minus 0.1 ms. In some embodiments, the injection duration is between about 2 ms and 2.4 ms. In some embodiments, the injection timing (e.g., the crank angle degree when the injector driver 136 sends the current waveform to the fuel injector 114) is at about 14.7 crank angle degrees plus or minus 0.1 crank angle degrees before top dead center (BTDC), which results in an SOI of about 13 crank angle degrees BTDC plus or minus 0.1 crank angle degrees (SOI=SOE+Constant), as shown in FIG. 10. In general, the injection timing and the injection duration described herein ensure that the fuel injection is completed prior to the piston 22 reaching TDC and aids in injection completing before the start of combustion (SOC). The mechanical fuel system used in the conventional ASTM D613 varies the injection duration based on the fuel properties and injection is not always completed before the SOC, which results in inconsistent combustion characteristics. The EFI system 14 provides more precise and repeatable combustion characteristics by completing injection prior to SOC, regardless of the physical properties of the fuel.

As shown in FIGS. 7A-9, the user interface 124 includes various screens that are displayed on the display 134. For example, the user interface 124 includes a main or setup screen 139 (FIG. 7A), a cetane test screen 140 (FIG. 7B), a fuel screen 142 (FIG. 8), and a graph screen 144 (FIG. 9). The various screen may be user-selectable via a user selecting a button (e.g., a digital button on the display 134) that corresponds with the particular display screen. The main screen 139 includes a cetane rating setup menu 145 (e.g., including one or more user-selectable icons or drop-down menus) that enables an operator to choose the fuel types, cetane numbers of the reference fuels, assign the fuel reservoirs 60 to a particular fuel type, the ASTM procedure being run (e.g., ASTM D613), etc. The main screen 139 also includes a begin rating button 147 (e.g., a digital button). In response to completing the test parameters via the cetane rating setup menu 145 and clicking on or touching the begin rating button 147, the user interface 124 may transition to the cetane test screen 140.

The cetane test screen 140 includes a cetane test table 146 that shows the order and data associated with a cetane number procedure (ASTM D613), including the fuel type, the fuel reservoir 60 (bowl) that corresponds with a particular fuel type being tested, and the results from recording data during the cetane number procedure (e.g., reading from the handwheel 28). In addition, the cetane test screen 140 displays the data inputs to the controller 126 and parameters calculated by the controller 126 that occur during the cetane test procedure described herein (e.g., SOI or ignition advance, and ignition delay).

The fuel screen 142 includes a diagram 148 of the EFI system 14 and indicates the operating conditions of the EFI system 14 to an operator (e.g., the open/close state of the fuel valve 62, the drain valves 64, and the flush valve 66, the pressure from the first pressure sensor 116, the pressure from the second pressure sensor 118, etc.). In some embodiments, the diagram 148 of the EFI system 14 is interactive and includes icons or buttons that are pre-programmed to provide input signals to the controller 126 that correspond with predetermined output control signals. For example, each of the fuel valves 62 (e.g., the first fuel valve 80, the second fuel valve 82, the third fuel valve 84, and the fourth fuel valve 86) may be represented as an icon in the diagram 148 and a user clicking on or touching (e.g., the display 134 may be a touchscreen) the icon for a particular one of the fuel valves 62 may transition that fuel valve 62 from the open position to the closed position, or vice versa. Similarly, each of the drain valves 64 (e.g., the first drain valve 100, the second drain valve 104, the third drain valve 106, and the fourth drain valve 108) and the flush valve 66 may be represented by an icon in the diagram 148 and a user clicking on or touching the icon for a particular one of the drain valves 64 or the flush valve 66 may transition that valve from the open position to the closed position or vice versa.

Alternatively or additionally, the fuel screen 142 may include one or more buttons (e.g., digital buttons) that instruct the fuel valves 62, the drain valves 64, and/or the flush valve 66 to open/close in a particular order. For example, the fuel screen includes a flush/fill bowl button 150 for at least a portion of the fuel reservoirs 60 (e.g., at least three of the fuel reservoirs 60), and a drain button 152 for at least a portion of the fuel reservoirs 60 (e.g., at least three of the fuel reservoirs 60. In some embodiments, in response to a user clicking on or touching one of the flush/fill bowl buttons 150, the controller 126 is configured to indicate a change in the fuel type in the corresponding fuel reservoir and an operator is prompted, via the user interface 124 (e.g., a visual indication) to add 100 mL of fuel to the corresponding fuel reservoir 60. Once the operator confirms, via interfacing with the user interface 124, that the 100 mL of fuel has been added to the corresponding fuel reservoir 60, the controller 126 instructs the drain valve 64 of the corresponding fuel reservoir 60 open for a predetermined amount of time (e.g., about 30 seconds) to drain the fuel from the corresponding fuel reservoir 60 and the corresponding first fuel filter. After the draining is completed, the operator is prompted, via the user interface 124 (e.g., a visual indication) to add the test fuel to the corresponding fuel reservoir 60. Once the operator confirms, via interfacing with the user interface 124, that the test fuel has been added to the corresponding fuel reservoir 60, the corresponding fuel reservoir 60 is ready to supply fuel to the cetane test engine 12.

In some embodiments, in response to a user clicking on or touching one of the drain buttons 152, the controller 126 instructs the drain valve 64 that corresponds with the fuel reservoir 60 being drained to actuate or move from the closed position to an open position. For example, if the drain button 152 associated with the first fuel reservoir 72 is activated, the controller 126 may instruct the first drain valve 100 to move from the closed position to the open position to allow the fuel from the first fuel reservoir 72 and the first fuel filter 90 to drain into the drain line 102. In some embodiments, the controller 126 may instruct the first drain valve 100 to remain open for a predetermined amount of time after the drain button 152 is activated and then automatically close the first drain valve 100 after the predetermined amount of time. In some embodiments, the controller 126 may instruct the first drain valve 100 to remain open until the drain button 152 is deactivated (e.g., clicked on or touched again).

The graph screen 144 includes a graphical display 154 of the current waveform that is supplied from the injector driver 136 to the fuel injector 114 and measured by the current sensor 138 as a function of crank angle degrees. In addition, the graph screen 144 includes a graphical display 156 of the pressure measured by the in-cylinder sensor 54 as a function of crank angle degrees, and a graphical display 158 of the first derivative of the pressure measured by the in-cylinder sensor 54 as a function of crank angle degrees. As shown in FIGS. 9 and 10, the controller 126 is configured to calculate this first derivative of the in-cylinder pressure and determine a crank angle degree location for the SOC as a constant offset (e.g., about 1 crank angle degree) from the crank angle degree associated with the maximum pressure rise rate on the in-cylinder pressure curve (e.g., where the first derivative defines a maximum value and the slope of a line tangent to the first derivative is zero). Accordingly, the controller 126 is configured to electronically receive data from the in-cylinder sensor 54 and calculate the SOC, which is used in the ignition delay calculation described herein.

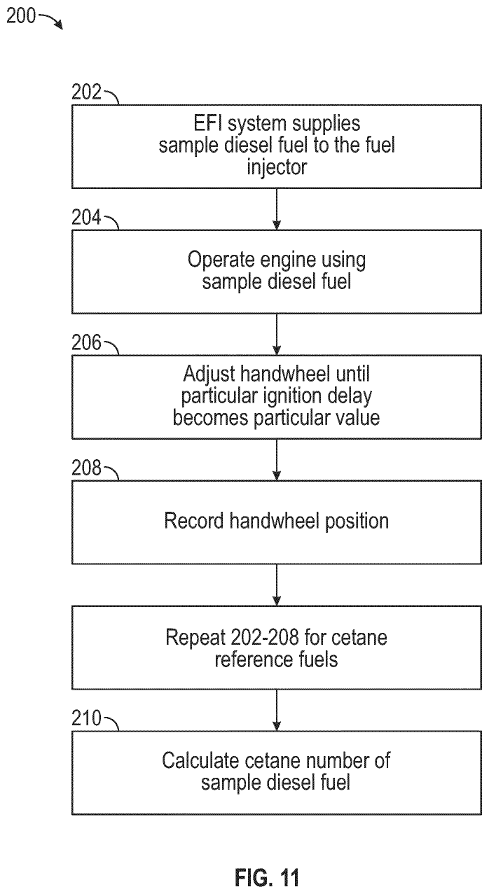

FIG. 11 illustrates an exemplary embodiment of a method, process or procedure 200 for determining a cetane number of a sample diesel fuel. In general, the method 200 follows the general procedure outlined in ASTM D613 for calculating the cetane number of a diesel fuel, but while utilizing the EFI system 14 and the HMI 16. In general, the electronic display, calculation, and control provided by the EFI system 14 and the HMI 16 significantly simplify and improve the precision of the method 200 relative to the mechanical fuel system implemented in ASTM D613. Per ASTM D613, a bracketing approach is used to determine the cetane number of a sample fuel relative to two reference diesel fuels of known cetane number (i.e., one low cetane reference fuel with a cetane number lower than the sample fuel and one high cetane reference fuel with a cetane number higher than the sample fuel). Accordingly, the method 200 may initiate, at step 202, where the EFI system 14 supplies one of the sample diesel fuel to the fuel injector 114 and to the cetane test engine 12. For example, in response to an operator clicking on or touching the begin rating button 147 on the main screen 139, the controller 126 may instruct the fuel valve 62 corresponding with the fuel reservoir 60 holding the sample diesel fuel to open and supply the sample diesel fuel to the fuel injector 114.

The cetane test engine 12 is then operated, at step 204, using one of the sample diesel fuel until equilibrium conditions are met and the ignition delay reaches a particular value in crank angle degrees. Specifically, the handwheel 28 is turned, at step 206, to adjust the compression ratio of cetane test engine 12 until the ignition delay reaches the particular value (13 crank angle degrees plus or minus 0.1 crank angle degrees, per ASTM D613), and the position of the handwheel 28 is recorded, at step 208, when the ignition delay reaches the particular value. The steps 202-208 are then repeated for the low cetane reference fuel and then for the high cetane reference fuel. The readings on the handwheel 28 for each of the sample diesel fuel, the low cetane reference fuel, and the high cetane reference fuel, and the known cetane numbers of the reference diesel fuels are used to calculate the cetane number of the sample diesel fuel, at step 210, per the equations in ASTM D613. In some embodiments another pass is conducted where the readings on the handwheel 28 are repeated (e.g., steps 202-208) in a fuel order corresponding to fuel reading sequence A of ASTM D613 (low cetane reference fuel->sample diesel fuel->high cetane reference fuel).

By incorporating the EFI system 14, the HMI 16, the in-cylinder sensor 54, and the crank angle encoder 56 into the cetane number rating system 10, several of the parameters required in the cetane rating procedure are electronically controlled, displayed, and/or calculated. For example, during operation of the cetane test engine 12, the controller 126 is configured to instruct the high-pressure pump 70 to maintain a constant fuel flow rate (e.g., about 11 mL/min) and instruct the fuel injector 114, via the injector driver 136, to maintain a constant SOI (e.g., 13 crank angle degrees before top-dead-center (BTDC) plus or minus 0.1 crank angle degrees, per ASTM D613) and a constant injection duration. The SOI is also verified and calculated based the current waveform measured by the current sensor 138, where SOE is detected and SOI is calculated as: SOI=SOE+Constant. The SOI calculated based on the measured current waveform may be displayed as “injection advance” on the cetane test screen 140 (see, e.g., FIG. 7B).

With the injection parameters held constant by the EFI system 14 and the HMI 16, an operator is not required to constantly adjust the injection parameters during the cetane number procedure and precision is substantially improved. Additionally, the controller 126 is configured to calculate the ignition delay as SOC minus SOI. As described herein, the controller 126 is configured to calculate the first derivative of the in-cylinder pressure and determine a crank angle degree location for the state of combustion (SOC) as a constant offset (e.g., about 1 crank angle degree) from the crank angle degree associated with the maximum pressure rise rate on the in-cylinder pressure curve (e.g., where the first derivative defines a maximum value and the slope of a line tangent to the first derivative is zero). Accordingly, the controller 126 is configured to calculate both SOC and SOI, and a difference between these terms in crank angle degrees determines the ignition delay. Per ASTM D613, the handwheel 28 is adjusted for each of the sample diesel fuel and both of the reference diesel fuels until the ignition delay reaches 13 crank angle degrees plus or minus 0.1 crank angle degrees. The controller 126 is configured to efficiently and precisely calculate the ignition delay, and display the calculated ignition delay to an operator on the cetane test screen 140 (see, e.g., FIG. 7B), which simplifies the cetane number procedure for the operator and produces more precise readings of the handwheel 28 (and thereby more precise calculations of the cetane number of the sample fuel).

In some embodiments, the EFI system 14, the HMI 16, the in-cylinder sensor 54, and the crank angle encoder 56 combine to form a retrofit kit that is used to retrofit conventional cetane number rating systems and replace the mechanical fuel systems with the EFI system 14 and the electronic control capabilities described herein.

As utilized herein with respect to numerical ranges, the terms “approximately,” “about,” “substantially,” and similar terms generally mean +/−10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the cetane number rating system 10 as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. An electronic fuel injection system for a cetane number rating system, the electronic fuel injection system comprising:
a fuel reservoir;
a supply line;
a drain line;
an electrically-controlled fuel valve configured to selectively provide or inhibit fluid communication between the fuel reservoir and the supply line;
an electrically-controlled drain valve configured to selectively provide or inhibit fluid communication between the fuel reservoir and the drain line;
a low-pressure pump in fluid communication with the supply line;
a high-pressure pump in fluid communication with an outlet of the low-pressure pump; and
an electronic fuel injector in fluid communication with an outlet of the high-pressure pump, wherein the high-pressure pump is configured to supply pressurized fuel from the fuel reservoir to the electronic fuel injector at a constant flow rate.

2. The electronic fuel injection system of claim 1, wherein the constant flow rate is 11 mL/min plus or minus 0.1 mL/min.

3. The electronic fuel injection system of claim 1, wherein the electronic fuel injector includes a plurality of nozzle holes, and wherein a portion of the plurality of nozzle holes are plugged so that fuel is injected from the electronic fuel injector through a portion of the plurality of nozzle holes that are unplugged.

4. The electronic fuel injection system of claim 1, further comprising an injector driver configured to supply a current waveform to the electronic fuel injector that defines a constant start of injection and a constant injection duration for the electronic fuel injector.

5. The electronic fuel injection system of claim 4, further comprising a current sensor configured to measure the current waveform supplied from the injector driver to the electronic fuel injector.

6. The electronic fuel injection system of claim 5, wherein the injector driver and the current sensor are in communication with a human-machine interface including a user interface and a controller.

7. The electronic fuel injection system of claim 6, wherein the user interface is configured to display the current waveform measured by the current sensor.

8. The electronic fuel injection system of claim 6, wherein the controller is configured to determine a start of injection for the electronic fuel injector based a start of energization of the current waveform and adding a constant to the start of energization.

9. The electronic fuel injection system of claim 6, wherein the electrically-controlled fuel valve and the electrically-controlled drain valve are controlled by the user interface.

10. A cetane number rating system, comprising:
a cetane test engine including a piston arranged within a cylinder;
an in-cylinder sensor configured to measure a pressure within a combustion chamber defined between the piston and the cylinder;
an electronic fuel injection system including an injector driver and an electronic fuel injector arranged to inject fuel into the combustion chamber; and
a human-machine interface in communication with the cetane test engine and the electronic fuel injection system, wherein the human-machine interface includes a user interface and a controller, the controller being configured to:
determine a start of injection for the electronic fuel injector by adding a constant to a start of energization of a current waveform supplied by the injector driver to the electronic fuel injector;
calculate a start of combustion based on a constant offset from a location of a maximum pressure rise rate of the pressure within the combustion chamber measured by the in-cylinder sensor; and
determine an ignition delay based on a difference between the start of combustion and the start of injection.

11. The cetane number rating system of claim 10, further comprising a low-pressure pump in fluid communication with a supply line, and a high-pressure pump in fluid communication with an outlet of the low-pressure pump.

12. The cetane number rating system of claim 11, wherein the electronic fuel injector is in fluid communication with an outlet of the high-pressure pump, and wherein the high-pressure pump is configured to supply pressurized fuel from the supply line to the electronic fuel injector at a constant flow rate.

13. The cetane number rating system of claim 12, wherein the constant flow rate is 11 mL/min plus or minus 0.1 mL/min.

14. The cetane number rating system of claim 11, further comprising an electrically-controlled fuel valve configured to selectively provide or inhibit fluid communication between a fuel reservoir and the supply line.

15. The cetane number rating system of claim 14, wherein the electrically-controlled fuel valve is controlled by the user interface.

16. The cetane number rating system of claim 10, further comprising a current sensor configured to measure the current waveform supplied from the injector driver to the electronic fuel injector.

17. The cetane number rating system of claim 16, wherein the user interface is configured to display the current waveform measured by the current sensor.

18. A method for determining a cetane number of a sample diesel fuel, the method comprising:
opening, in response an input to a user interface, a fuel valve to supply a sample diesel fuel to a supply line;
pumping the sample diesel fuel from the supply line to an electronic fuel injector at a constant flow rate;
instructing, via an injector driver, the electronic fuel injector to inject the sample diesel fuel into a cetane test engine;
measuring, via a current sensor, a current waveform supplied by the injector driver to the electronic fuel injector;
calculating a start of injection by:
determining a start of energization from the current waveform; and
adding a constant to the start of energization;
measuring, via an in-cylinder sensor, a pressure within a combustion chamber of the cetane test engine;
calculating a start of combustion in the cetane test engine based on a constant offset from a location of a maximum pressure rise rate of the pressure measured by the in-cylinder sensor;
calculating an ignition delay based on a difference between the start of combustion and the start of injection;
turning a handwheel on the cetane test engine until the ignition delay becomes 13 crank angle degrees plus or minus 0.1 crank angle degrees; and
recording a position of the handwheel when the ignition delay is 13 crank angle degrees plus or minus 0.1 crank angle degrees.

19. The method of claim 18, wherein pumping the sample diesel fuel from the supply line to the electronic fuel injector at the constant flow rate comprises:
pumping, via a low-pressure pump, the sample diesel fuel from a fuel reservoir to the supply line; and
pumping, via a high-pressure pump, the sample diesel fuel from the supply line to the electronic fuel injector.

20. The method of claim 19, wherein the constant flow rate is 11 mL/min plus or minus 0.1 mL/min.
